(19) United States
(12) Patent Application Publication
(10) Pub. No.: US 2025/0041662 A1
(43) Pub. Date: Feb. 6, 2025

(54) WEARABLE DEVICE FOR MEASURING SPORTS PERFORMANCE

(71) Applicant: TALENTPLAYERS S.R.L., Palermo (IT)

(72) Inventors: Antonio DI STEFANO, Palermo (IT); Vincenzo LI VIGNI, Palermo (IT); Fabrizio BUCCHERI, Palermo (IT)

(21) Appl. No.: 18/718,042

(22) PCT Filed: Dec. 7, 2022
(86) PCT No.: PCT/IB2022/061870

§ 371 Date: Jun. 9, 2024

(30) Foreign Application Priority Data
Dec. 10, 2021 (IT) 102021000031094

(51) Int. Cl. A63B 24/00

(52) U.S. Cl. CPC A63B 24/0062; A63B 24/0006; A63B 2220/803; A63B 2220/836

(57) ABSTRACT

A wearable device for measuring sports performance comprises a wearable containment casing which houses therewithin a PCB provided with an electronic circuit having a microcontroller (MCU), movement detection means suitable to detect data relating to the instantaneous value of accelerations and rotation speed and to transfer them to said microcontroller for their processing thereby, wherein the microcontroller is suitable of simultaneously executing three secondary algorithms for calculating three different instantaneous speeds (V1(t), V2(t), V3(t)) to calculate the instantaneous speed (v(t)) as the weighted average of the values of said instantaneous speeds of said three secondary algorithms, using the formula

$$v ( t ) = a * V 1 ( t ) + b * V 2 ( t ) + c * V 3 ( t )$$

wherein a, b and c are the three coefficients of the weighted average selected as a function of the detected step frequency.

### TECHNICAL FIELD

The present invention finds application in the field of athletic performance monitoring devices and particularly relates to a wearable device for measuring an athlete's sports performance to collect useful data for evaluating effort, energy expended and physical stress suffered by the athlete during the activity, also for the purpose of regulating the intensity of training and therefore maximizing or evaluating its effectiveness and the performance achieved in various activities.

### STATE OF THE ART

The known wearable devices for measuring performances of an athlete generally use technologies based on GPS data collection, or similar technologies such as LPS (Local Positioning System) or other technologies based on local radio references, suitable to detect the movement of the athlete.

In this way, it is possible to record the movement data for obtaining data such as instantaneous speed, accelerations, changes of direction and to calculate the metabolic data relating to the expended energy and power.

However, GPS-based systems, at least those in the medium or low cost range, are not very precise, while much more complex and expensive devices have to be used to have greater reliability.

Also, they can only be used outdoors, requiring good sky visibility to work.

Furthermore, GPS devices require several minutes to synchronize with the satellite signal, must be placed on the back, are physically quite large and therefore potentially dangerous in case of injury.

Last but not least, the movements detected by these systems are indirect, being calculated on the basis of satellite data and not on the athlete's real movements.

LPS systems may also operate indoors and are much more accurate, but also much more expensive and require the installation of several transmitters around the operation area, making them impractical and, therefore, little used.

Recently, some GPS-based devices have started to use inertial sensors (MEMS) to correct GPS data inaccuracies, but up to now there are no devices, technologies or algorithms on the market for obtaining complete and sufficiently precise data from inertial sensors alone.

The only other currently known alternative to GPS/LPS systems are video analysis based measurement systems, consisting of several cameras mounted around the operation area and dedicated analysis software. However, these systems are rarely used because they are impractical and extremely expensive.

A further drawback of the solutions based exclusively on inertial sensors currently on the market is related to the intrinsic imprecision of the commonly used algorithms which do not work well in real situations wherein the movements are not clean, but potentially affected by inaccuracies and external factors and in some cases are difficult to apply in real time due to their complexity.

The algorithms traditionally employed on the best known inertial systems and applied to signals relevant to the legs are essentially of two types.

A first typology is based on the kinematics of the movement of the leg and calculates the distance walked in a single step by integrating the acceleration in the direction of movement twice, with the first integration providing a velocity vector, while the second provides a displacement vector.

Knowing the time in which the step has been completed, it is also possible to obtain the speed of the single step from the distance and from this the average speed of the athlete. This type of algorithm suffers heavily from the problem of accumulation of errors due to integrations. As matter of fact, each integration, due to this effect, produces a signal that diverges over time and which, therefore, is not usable.

The method commonly used to limit this effect consists in forcing the speed value to zero when the foot is on the ground.

This event can be derived from the leg tilt, detected by the signal of the gyroscope.

This type of algorithm is called ZUPT (Zero velocity UPdaTe) in the literature and provides discrete precision especially in low-speed movement (walking, but not running), if there are no external interferences in the movement.

The second type of algorithms for estimating the velocity of the center of mass are those based on empirical methods.

The most common ones derive from the assumption that the movement of the leg is similar to that of a pendulum, and use the number of steps taken and the measurement of the time between one step and the next to derive distance and speed according to empirical factors.

For example, one of the simpler methods assumes that the average step is about 100.74 m long. The number of steps, multiplied by this value, therefore provides the total distance, while the time in which a step is taken, provides the instantaneous speed. This model can be refined by considering different step lengths as a function of step frequency (more frequent steps correspond to running with longer steps).

This class of algorithms provides reasonable performance, even in the presence of interference, but requires calibration, because it is affected by systematic errors such as the influence of the athlete's height, and therefore of his legs, and the specific walking or running style.

Finally, there are other slightly different empirical algorithms that apply to signals recorded in the upper part of the body (belt, back, wrist). These algorithms consider, for example, the amplitude of the accelerations at each step and their functions, for example the fourth root. However, even these algorithms do not provide satisfactory measurements, in all conditions.

### SCOPE OF THE INVENTION

The object of the present invention is to overcome the above drawbacks by providing a wearable device for measuring sports performance which combines high reliability in the measurements with relatively cost-effectiveness.

A particular object is to provide a wearable device for measuring sports performance which does not require external signals, thus also being adapted to be used indoors.

Still another object is to provide a wearable device for measuring sports performance which is relatively small and light and therefore not very invasive.

Yet another object is to provide a wearable device for measuring sports performance which can detect and measure specific movements of the athlete which GPS cannot detect, such as jumps, shots, falls and the like.

Still another object is to provide a wearable device for measuring sports performance which allows more extensive and varied applications than those possible with GPS, while providing at least the same data, such as monitoring of physical stress and injury prevention.

Yet another object is to provide a wearable device for measuring sports performance which uses an algorithm capable of providing precise results and which is also computationally efficient so as to be applied in real time by the microcontroller used to control the device.

These objects, as well as others which will become more apparent hereinafter, are achieved by a wearable device for measuring sports performance which, according to claim 1, comprises a wearable containment casing which houses thereinside a PCB provided with an electronic circuit having a microcontroller (MCU), movement detection means comprising at least one inertial sensor suitable for detecting data relating to the instantaneous value of the accelerations on three predetermined orthogonal reference axes and to the rotation speeds around said reference axes and for transferring them to said microcontroller for their processing thereby, wherein said microcontroller is adapted to execute at least one first main algorithm for acquiring, processing and communicating said data to obtain the instantaneous speed referred to the user's center of mass and the changes of direction thereof.

In a peculiar way, the microcontroller is suitable for simultaneously executing three secondary algorithms for the calculation of three different instantaneous speeds (V1(t), V2(t), V3(t)) and wherein said first main algorithm is suitable for calculating said instantaneous speed (v(t)) as the weighted average of the values of said instantaneous speeds of said three secondary algorithms, using the formula

v(t)=a*V1(t)+b*V2(t)+c*V3(t)

wherein a, b and c are the three coefficients of the weighted average selected according to the detected frequency of the steps and having respective values such that their sum is equal to 1 and wherein said secondary algorithms have respective measurement precisions differentiated from each other according to a specific speed regime.

The model thus obtained is also surprisingly independent of the physical and athletic properties of the athlete, thanks also to the use of an exclusively inertial system to collect the same data traditionally provided by GPS or LPS systems.

The device differs from all these systems in the following features:
it is suitable to measure the movement directly, using a MEMS inertial sensor comprising an accelerometer and a tri-axial gyroscope;
does not require external signals, so it can also be used indoors;
it is smaller and lighter than a GPS receiver and therefore less invasive;
it can detect and measure specific movements of the athlete that GPS cannot detect, such as jumps, throws, falls;
it allows more extensive and varied applications than those possible with GPS, for example the monitoring of physical stresses and the prevention of accidents;
it is constructively simpler, and therefore less expensive than other technologies.

Advantageous embodiments of the invention are obtained according to the dependent claims.

### BRIEF DISCLOSURE OF THE DRAWINGS

Further features and advantages of the invention will become more apparent in the light of the detailed description of a preferred but not exclusive embodiment of the device according to the invention, shown by way of non-limiting example with the aid of the attached drawings wherein:

FIG. 1 is a schematic view of a circuit diagram of the device according to the invention;

FIG. 2 is a diagram schematically illustrating the operation of the first main algorithm.

### BEST MODES OF CARRYING OUT THE INVENTION

The device according to the present invention essentially consists of a small wearable device which, used together with suitable software, allows to accurately measure an athlete's physical activity and performance during training or competition, in order to collect useful data to evaluate the effort, the energy expended and the physical stress suffered by the athlete during the activity, to regulate the intensity of the workouts and therefore maximize or evaluate their effectiveness and the performances achieved in various activities.

The device may be used in both individual and team sports and is generally made to be worn by the athlete before the start of the activity, is activated and then recovered at the end of the activity.

The data are normally recorded in the memory of the device itself and downloaded at the end, but they may also be transmitted in real time via radio if required, to be viewed and analyzed with special software and platforms that are part of the whole system.

Preferably, the device is worn on the leg, just above the ankle, and may be integrated into clothing or game equipment such as for example shin guards, in the case of soccer. Therefore, it is not very invasive, not very dangerous, and is in a position where it can directly measure movements of primary interest.

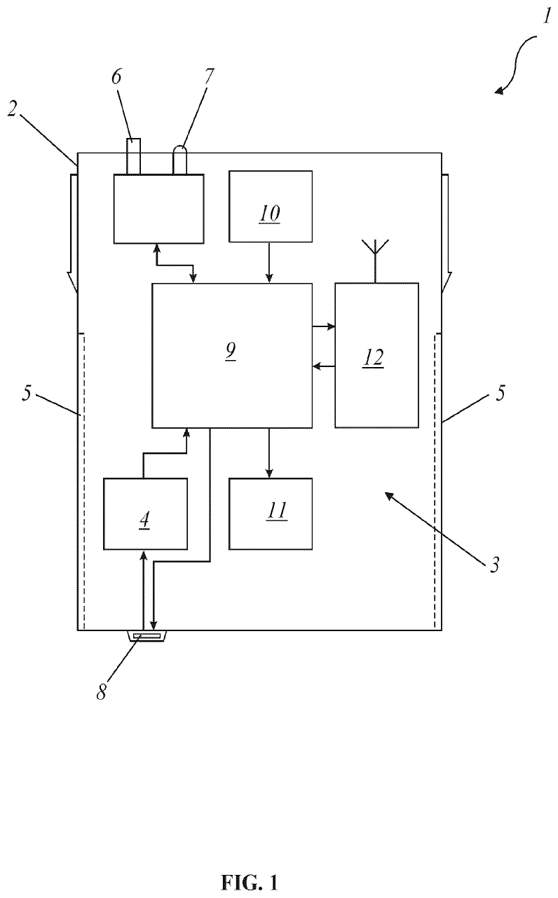

According to a preferred but not exclusive embodiment, schematized in FIG. 1, the device according to the invention, globally indicated with 1, comprises a casing 2 with small size (about 45×20×8 mm) and low weight (less than 10 g), preferably in plastic and/or elastomeric material to be non-conductive and to allow data exchange with the outside via radio.

The casing 2 will preferably be made of impact-proof plastic material, such as ABS or the like, or of an elastomeric material (rubber or silicone) or a mixture of the two (i.e. “overmold”), suitably closed to prevent the entry of water and dust and will contain an electronic circuit, globally indicated with 3, and a battery 4.

Conveniently, the casing 2 may have a flattened shape with convex surfaces and rounded edges so as not to cause discomfort to the athlete wearing it and not to create dangers in the event of impacts or accidents.

The outer wall may integrate guides 5, such as grooves, recesses or joints, which allow it to be fixed to sports equipment or clothing provided with particular hooks or joints. Furthermore, it may be equipped with at least one button 6 or actuating surface, with a light indicator (LED) 7 and with an electrical connection port 8 for recharging and/or exchanging data.

By way of example, the device 1 may be worn by inserting it into a specific elastic band equipped with a pocket with Velcro ends, or by inserting and fixing it by means of the special guides 5 in a suitable support sports equipment, not illustrated, such as a shin guard equipped with compartment with compatible guides and joints.

The electronic circuit 3 located inside the casing 2 comprises various subsystems. One of them is dedicated to power management and comprises voltage regulators, solid-state switches, battery charging circuits 4 and charge metering.

This part shall be designed to enable efficient use of energy to maximize the life of the battery 4 and enable the use of a battery 4 with smaller size.

The second subsystem is represented by the microcontroller MCU 9 and its inner and outer peripherals. This part coordinates the operation of the remaining subsystems and is responsible for executing the data acquisition, processing and communication algorithms.

Among the peripherals controlled by the MCU is the RGB LED 7 which is used to signal the user the operating mode and the push button 6, mechanical, capacitive or otherwise, used to control the operating modes.

The motion detection subsystem comprises a MEMS (MicroElectroMechanical System) inertial sensor 10 provided with at least one tri-axial accelerometer and one tri-axial gyroscope for a total of 6 degrees of freedom (6 DOF).

Eventually, the MEMS sensor 10 will integrate an intelligent pre-processing portion, comprising, for example, frequency filtering of the data, pattern detection based on programmable state machine or machine learning/neural network.

The sensor 10 will be connected to the MCU 9 which reads the data and uses them for movement analysis through the proprietary algorithms described below.

The memory 11 consists of a rewritable and sufficiently fast non-volatile memory, typically Flash, EEPROM, MRAM or the like. Its size is such as to allow data storage of at least two hours of use.

Memory 11 will also be connected to MCU 9 which writes the processed data and reads them in the communication stage with the outside.

A further subsystem is the radio subsystem 12, used by the MCU 9 to receive commands from an external communication device, such as a smartphone, a tablet, a PC or the like, to send the acquired or recorded data.

Typically, but not exclusively, the radio system 12 will operate in the 2.4 GHz ISM band and will be adapted to communicate using proprietary protocols or the standard Bluetooth Low Energy protocol (BLE, version 4.2, 5.0 and later).

The antenna of the radio system 12 may be integrated in the same PCB or printed circuit comprising the electronic circuit 3 and, therefore, is contained inside the plastic casing 2.

All subsystems are optimized for low power operation and therefore may be deactivated when not in use to limit power consumption.

The used battery 4 may be of both the primary and secondary type, i.e. rechargeable. In the first case it may have a voltage of at least 3 V and a capacity of at least 200 mAh, while in the second case it may be a single cell lithium ion (Li-ion) battery (3.7 V) with a capacity of approximately 50 mAh.

If the battery 4 is rechargeable, the energy will be powered from the outside through a suitable connector 8, which may consist of simple exposed metal pads, which may be coupled with “pogo-pins”, or be a standardized and miniaturized connector, such as for example the Micro-USB.

In both cases there may be only the connections for the power supply or also others for data exchange. In some implementations the power may be supplied via electromagnetic coupling, therefore without the need for contact.

From an operational point of view, the device 1, when turned on, may be in three different states: standby, recording and recharging.

The first two states may be selected by pressing button 6 on the casing 2, while the third state is selected automatically when an energy source is connected.

In the standby state, the electronic circuit 3 activates the radio system 12 and may receive commands from the outside by means of a fixed or movable communication device, such as a PC, smartphone or tablet.

Commands allow you to start logging, clear memory, sync time, read status or data, and shut down the device 1.

The actual operation is that of the recording state, in which device 1 acquires, processes and records the data relating to the athlete's movement.

In this state, the device 1 acquires data from the MEMS sensor 10 with a sampling frequency between 10 and 1000 Hz, preferably 100 Hz. These data include the instantaneous value of the accelerations on the three orthogonal axes Ax, Ay and Az, and the speeds of rotation around the same axes Gx, Gy and Gz.

These raw data referring to the part of the body to which the device 1 is anchored, for example one of the legs, are processed in real time by the MCU 9 according to proprietary algorithms described below so as to obtain the instantaneous speed v(t) referring to the center of mass of the athlete and his/her changes of direction.

The data is decimally reduced by a programmable factor, generally from 1 to 50 Hz, preferably from 1 to 10 Hz, and recorded in the non-volatile memory.

The data recorded in this way is sufficient to describe the macroscopic movement of the athlete and allow to obtain other data, such as instantaneous accelerations, deriving speed data, total distance, integrating speed data, speed distribution statistics, statistics on direction changes and metabolic data, which may be calculated from the other data using well-known and very common models, such as the Di Prampero-Osgnach model.

In addition to this first main algorithm, the device runs some additional proprietary auxiliary algorithms to detect and measure some significant events such as jumps, throws, bumps, falls.

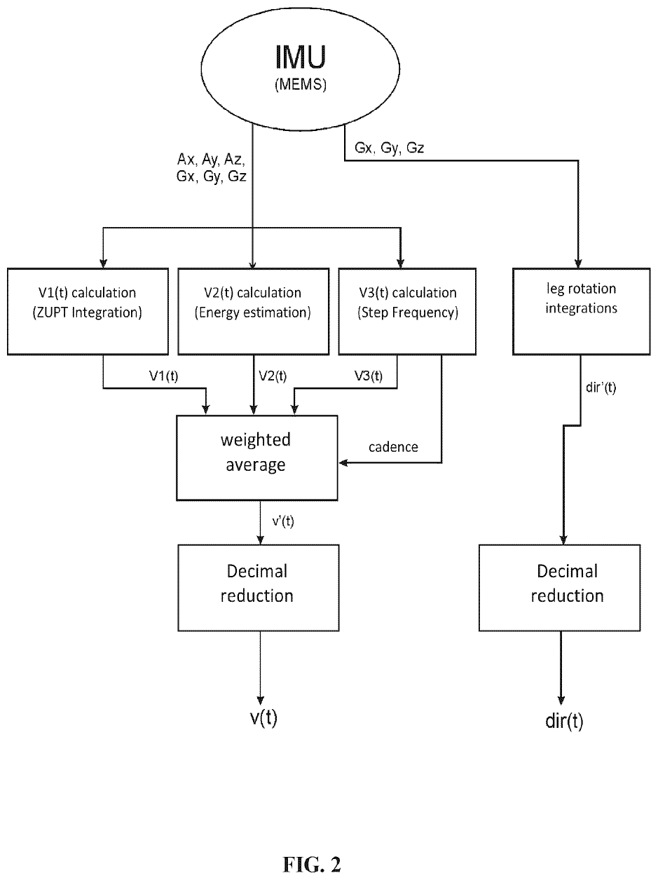

FIG. 2 schematises the operating mode of the first main algorithm, which is essentially based on the weighted average of three different secondary algorithms, each functioning better than the others in a specific speed regime.

The weight of the three secondary algorithms is therefore modified according to the frequency of the steps detected.

The three secondary algorithms used are as follows:

1) a first simplified ZUPT algorithm, wherein every single step is identified and the acceleration along the front-back axis is integrated only in the time in which the foot is not in contact with the ground, while in the remaining interval the speed is set to zero. Further integration of this signal gives the distance walked in one step. From the knowledge of the duration of the step (which may be obtained from the number of samples and the sampling frequency), it is possible to calculate the average speed during the step V1(t). This speed is time-averaged to provide a more continuous signal and filter out any interference in the movement;

2) a second secondary algorithm that derives from energy considerations on the movement: a first order low-pass filter is applied to the acceleration vector module with a time constant of the order of the second (for example from 0.5 to 2 seconds), subtracted the module of the vector of gravity, and from the resulting value calculated the instantaneous speed through a function of the type

$$V 2 ( t ) = f * ( A ( t ) - g ) \hat{} h / k ,$$

wherein V2(t) is the resulting instantaneous speed, A(t) is the modulus of the acceleration vector to which the low-pass filter has been applied, g is the modulus of the gravity vector, which may be set during the calibration step or obtained dynamically, while h and k are two real numbers obtained experimentally between 0.5 and 1.5 for h and 1.0 and 2.0 for k and which depend on the shape and position of the device. The factor f, on the other hand, is used to convert the numerical units supplied by the sensor into m/s, and in this specific case it is equivalent to approximately 1/5000;

3) a third secondary algorithm of empirical type for the calculation of the respective instantaneous speed V3(t), based on the counting of the steps, their cadence and on a variable length of the step tabulated as a function of the cadence.

The three algorithms are executed simultaneously and the resulting final speed is calculated as:

$$v ( t ) = a * V 1 ( t ) + b * V 2 ( t ) + c * V 3 ( t )$$

wherein Vi(t) are the speeds provided by the three algorithms, while a, b and c are the three coefficients of the weighted average, such that the sum a+b+c=1.

The coefficients a, b and c may be selected according to different strategies and according to the applications. In some implementations, their value is fixed and equal to ⅓, in other implementations different values are used for different speed zones, for example:
for v(t−1)<3 m/s the following are used: a=½, b=½, c=0
for 3 m/s>=v(t−1)<6 m/s the following are used: a=⅓, b=⅓, c=⅓
for v(t−1)>=6 m/s the following are used: a=0, b=½, c=½

In some implementations two of the coefficients a, b or c may be set to 0, so that only one of the three algorithms is used.

The data v(t), thus calculated, is obtained with the same sampling frequency of the sensor 10. Generally, this is excessive for real applications, therefore these data ongoing to a decimal reduction (typically by a factor from 4 to 100, i.e. up to 1-25 Hz) before being saved in memory. In this way, memory occupation and data download time are reduced, without causing a loss of precision.

It has also been verified experimentally that the instantaneous speed data thus obtained is so accurate that to obtain the total distance travelled, it is sufficient to integrate the data processed with decimal reduction in time a posteriori.

Repeated tests carried out with different athletes resulted in an average error on the estimated speed of less than 0.1 m/s with a standard deviation of ±0.2 m/s.

The direction changes may be obtained from the data provided by the gyroscope, acquired at the same sampling frequency as the accelerations, which are more precise than the integration of the lateral acceleration. However, this operation is not obvious because the gyroscope data is affected by two problems:
during the movement of the leg the axes of the gyroscope around which the rotation occurs alternate between two directions, depending on the position of the leg;
the rapid and continuous movements of the leg cause an unpredictable drift of the gyroscope signals, which therefore cannot be directly integrated.

Since there are no known solutions in the literature for both of these problems, and since there is not even an unambiguous and analytical definition of the intuitive concept of “change of direction”, the device 1 also uses a second proprietary algorithm in this case to obtain sufficiently precise data.

The algorithm consists in obtaining the modulus of the rotation of the vector around the longitudinal axis, applying to this a high-pass filter with a time constant of the order of 5-10 seconds to remove the drift, and integrating the resulting signal to obtain the relative angle.

The signal thus obtained is decimally reduced by the same factor used for the instantaneous speed and saved in memory.

Single direction changes can be calculated a posteriori by detecting discontinuities (jumps) in this relative direction signal: these will be positive for angles to the right and negative to the left.

In addition to the main algorithms for calculating instantaneous speed and direction (angle), the device comprises some auxiliary algorithms which are optionally used in some versions or for some specific applications.

A first auxiliary algorithm is that relating to the detection and measurement of jumps. Its operation is based on the identification of intervals of predetermined minimum duration, for example at least 0.1-0.5 seconds, in which all three accelerations Ax, Ay and Az result simultaneously with a modulus lower than 0.1 g.

The height of the jump is determined by the duration of this interval, which corresponds to the free fall time.

A second auxiliary algorithm is used to detect throws (kicks) and is based on the detection of an interval of minimum predetermined duration, for example at least 0.1 seconds, wherein the module of the acceleration vector is greater than a predetermined threshold value, for example 8 g.

The force of the pull may be determined by integrating the acceleration vector in the interval in which it is greater than the threshold value.

A third auxiliary algorithm is that relating to the detection of falls and is based on the detection of the change of axis on which the gravity vector manifests itself for a time greater than 1 second.

Finally, a further auxiliary algorithm estimates the load (and therefore the stress) on the tibia and on the related joints (knee and ankle). This algorithm simply accumulates the modulus of acceleration along the axis parallel to the tibia.

The device 1 according to the present invention may be integrated into a complete system which allows data to be acquired, shared, saved, catalogued, processed and displayed.

The whole system comprises software, typically an application for smartphones, tablets or PCs which allows interaction with the device 1. The interfacing takes place via the radio interface 12, generally Bluetooth LE, or optionally other types of protocols. The application allows you to send some commands to device 1, such as precise time synchronization, start recording, erase memory and switch off, and download the recorded data.

In some implementations, decimally reduced data is sent in real time via radio, and then viewed and saved directly by the software.

The application is also used to send the downloaded data to a centralized remote server, which contains further tools for analysis and visualization in the form of a web-app. The server has the function of data storage, organization, post-processing and display. In particular, by accessing the server via the web, it is possible to retrieve data from any location and view its processing. These consist in particular of statistical analyzes that better highlight some salient features relating to athletic performance.

Since these analysis algorithms are applied retrospectively, it is possible to personalize them, also according to the sport considered.

1. A wearable device for measuring sports performance, comprising:
a wearable containment casing (2) housing thereinside a PCB provided with an electronic circuit (3) having a microcontroller (9);
motion detection means comprising at least one inertial sensor (10) adapted to detect data relating to the instantaneous value of the accelerations on three predetermined orthogonal reference axes and to the rotation speeds around said three predetermined orthogonal reference axes and to transfer said data to said microcontroller (9) for the processing of said data by said microcontroller (9);
wherein said microcontroller (9) is adapted to execute at least one first main algorithm for acquisition, processing and communication of said data to obtain the instantaneous speed referred to the user's center of mass and the changes of direction of said center of mass;
wherein said microcontroller (9) is adapted to simultaneously carry out three secondary algorithms for the calculation of three different instantaneous speeds (V1(t), V2(t), V3(t)) and wherein said first main algorithm is suitable to calculate said instantaneous speed (v(t)) as a weighted average of the values of said instantaneous speeds of said three secondary algorithms, by means of the formula
$$v ( t ) = a * V 1 ( t ) + b * V 2 ( t ) + c * V 3 ( t )$$
wherein a, b and c are the three coefficients of the weighted average selected as a function of the detected step frequency and wherein said secondary algorithms have respective measurement accuracies differentiated from each other as function of a specific speed regime.

2. The wearable device as claimed in claim 1, wherein a first of said secondary algorithms is a simplified ZUPT (Zero velocity UPdaTe) algorithm.

3. The wearable device as claimed in claim 2, wherein a second of said secondary algorithms is an algorithm wherein a first-order low-pass filter with a time constant of the order of second is applied to the modulus of the acceleration vector, than subtracted the magnitude of the gravity vector, and the instantaneous velocity is calculated from the resulting value using the function:
$$V 2 ( t ) = f * ( A ( t ) - g ) \hat{} h / k ,$$
wherein V2(t) is the resulting instantaneous velocity, A(t) is the modulus of the acceleration vector to which said low-pass filter has been applied, g is the modulus of the gravity vector, h and k are two real numbers obtained experimentally and wherein f is a conversion factor of the numerical units provided by said inertial sensor (10).

4. The wearable device as claimed in claim 3, wherein said time constant has a value comprised within 0.5-2 s and wherein said real numbers h and k have respective values which depend on the shape and position of the wearable device and which are comprised respectively between 0.5 and 1.5 and between 1.0 and 2.0.

5. The wearable device as claimed in claim 4, wherein the third of said secondary algorithms is an empirical algorithm for the calculation of the respective speed V3(t) based on the count of steps, on their frequency and on a variable length of the step as a function of the frequency.

6. The wearable device as claimed in claim 1, wherein said microcontroller (9) is programmed to decimally reduce the calculated data by a programmable factor and for acquiring said data from said inertial sensor (10) with a sampling frequency comprised between 10 and 1000 Hz, preferably equal to 100 Hz.

7. The wearable device as claimed in claim 1, wherein said inertial sensor (10) is a MEMS (MicroElettroMechanical System) inertial sensor provided with at least one tri-axial accelerometer and a tri-axial gyroscope for a total of 6 degrees of freedom (6 DOF) and integrating an intelligent pre-processing portion, comprising data rate filtering, programmable state machine or machine learning-/neural network-based pattern detection.

8. The wearable device as claimed in claim 1, wherein said electronic circuit (3) comprises a radio subsystem (12) used by said microcontroller (9) to receive commands from an external communication device and to send the detected data.

9. The wearable device as claimed in claim 8, wherein said microcontroller (9) is adapted to carry out a second main algorithm for the calculation of direction changes and which consists in obtaining the modulus of the rotation of the vector around to the longitudinal axis, applying a high-pass filter with a time constant of the order of 5-10 seconds thereto to remove the drift, integrating the resulting signal to obtain the relative angle and wherein said resulting signal is decimally reduced by the same factor used for said instantaneous speed (v(t)).

10. The wearable device as claimed in claim 1, wherein said microcontroller (9) is adapted to carry out a first auxiliary algorithm for the detection and measurement of jumps which is based on the identification of intervals of at least 0.1-0.5 seconds wherein all three accelerations (Ax, Ay, Az) around said three orthogonal axes simultaneously result in modulus lower than a predetermined value, a second auxiliary algorithm for detecting shots and which is based on the detection of an interval of at least 0.1 seconds wherein the magnitude of the acceleration vector is greater than a predetermined value, a third auxiliary algorithm for detection of falls which is based on the detection of the change of axis on which the gravity vector manifests itself for a time greater than a predetermined time, a fourth auxiliary algorithm for estimating the load on the tibia and its joints and which is based on the accumulation of acceleration module along the axis parallel to the tibia.

11. The wearable device as claimed in claim 6, wherein said programmable factor is comprised between 1-50 Hz.

12. The wearable device as claimed in claim 6, wherein said programmable factor is comprised between 1-10 Hz.
